(19) United States
(12) Patent Application Publication
(10) Pub. No.: US 2024/0091040 A1
(43) Pub. Date: Mar. 21, 2024

(54) ORTHOTIC ANKLE SUPPORT DEVICE

(71) Applicant: RHEOHERO, INC., Ithaca, NY (US)

(72) Inventors: LUDOVICO CESTAROLLO, Ithaca, NY (US); KARAN VISHWANATH, Ithaca, NY (US)

(73) Assignee: RHEOHERO, INC., Ithaca, NY (US)

(21) Appl. No.: 18/468,388

(22) Filed: Sep. 15, 2023

Related U.S. Application Data
(60) Provisional application No. 63/375,908, filed on Sep. 16, 2022.

(51) Int. Cl. A61F 5/01

(52) U.S. Cl. CPC A61F 5/0111

(57) ABSTRACT

An orthotic ankle support system having a compression sleeve having an interior space to receive an ankle. Further included is a double strap having a midline; a first strap having alternated inelastic segments and elastic segments, a first inelastic segment extending from the midline towards a last inelastic segment at a free end; a second strap extending from the midline opposite the first strap, the second strap having alternated inelastic segments and elastic segments, a first inelastic segment extending from the midline towards a last inelastic segment at a free end. The midline is positioned on a posterior side of the compression sleeve having the first strap and the second strap wrapped in opposite directions around the compression sleeve and adjusted to vary tension to hold and grip the ankle.

### CROSS REFERENCE

This Application claims the benefit of priority under 35 U.S.C. § 119(e) to U.S. Provisional Patent Application Ser. No. 63/375,908 filed Sep. 16, 2022, the contents of which are incorporated by reference herein as if set forth in their entirety for all purposes as if put forth in full below.

### BACKGROUND OF THE INVENTION

### Technical Field

This invention relates broadly to joint support systems, devices, and methods. More particularly, this invention relates to tension adjustable orthotic ankle support systems, devices, and methods.

### Background Information

Lower body injuries, particularly ankle sprains, represent one of the most common injury concerns for athletes. As a preventative measure, it is common for athletes to get their ankles taped and/or braced prior to practices and games. In sports such as basketball, tape is commonly used to secure the ankles of the athletes. This is because taping is slim and conforms well to the size and shape of the ankle, while not excessively restricting the range of motion. However, taping is also very time consuming and wasteful, and tape loses effectiveness over time as it is worn.

An alternative to ankle tape are ankle braces. Braces are fast and easy to apply and are known to well protect the ankle over time. However, braces are bulky, and athletes find them uncomfortable.

There is a need for athletes to have an effective ankle support solution that is slim and flexible, can be applied quickly, and minimizes range of motion constraints.

### SUMMARY OF THE INVENTION

The present disclosure is directed toward systems, methods, devices, providing orthotic ankle support.

In one aspect of the present disclosure provided herein, is an orthotic ankle support system having a compression sleeve having an interior space to receive and conform to a foot, an ankle, and a portion of a lower leg of a person. Further included is a double strap having a midline; a first strap having alternated inelastic segments and elastic segments, a first inelastic segment extending from the midline towards a last inelastic segment at a free end, and having a plurality of alternated elastic segments and inelastic segments, therebetween; a second strap extending from the midline opposite the first strap, the second strap having alternated inelastic segments and elastic segments, a first inelastic segment extending from the midline towards a last inelastic segment at a free end, and having a plurality of alternated elastic segments and inelastic segments therebetween. The first strap and the second strap extend from the midline in opposing directions with the midline double strap positioned on a posterior side of the compression sleeve, having the first strap and the second strap wrapped in opposite directions around the compression sleeve and fastened to each other and adjusted to vary tension to hold and grip the foot and ankle, when inserted into the sleeve.

In another aspect of the present disclosure provided herein, is a method for applying an orthotic ankle support device, including applying a compression sleeve to a foot, an ankle, and a lower leg, the compression sleeve having a double strap connected at a midline to a posterior facing section of the compression sleeve. The method further includes moving a first strap about a high lateral section of the ankle; moving the first strap over a bridge of the foot towards a medial side of the foot; moving the first strap under an arch of the foot towards a lateral side; moving the first strap over the bridge of the foot towards a high medial section of the ankle; holding the first strap to the midline of the double strap; moving a second strap section about the high medial section of the ankle; moving the second strap over the bridge of the foot towards the lateral side of the foot; moving the second strap under the arch of the foot towards the medial side; moving the second strap over the bridge of the foot towards a high lateral section of the ankle; adjusting the strap tension; and affixing the second strap to the first strap.

In another aspect of the present disclosure provided herein, is an orthotic ankle support device having a double strap including a midline; a first strap having alternated inelastic segments and elastic segments, a first inelastic segment extending from the midline towards a last inelastic segment at a free end; and a second strap extending from the midline opposite the first strap, the second strap having alternated inelastic segments and elastic segments, a first inelastic segment extending from the midline towards a last inelastic segment at a free end. The double strap is positioned around an ankle having the first strap section and the second strap section wrapped in opposite directions and adjusted to vary tension to hold and grip the ankle.

These, and other objects, features and advantages of this invention will become apparent from the following detailed description of the various aspects of the invention taken in conjunction with the accompanying drawings.

### BRIEF DESCRIPTION OF THE DRAWINGS

The present invention will be understood more fully from the detailed description given hereinafter and from the accompanying drawings of the preferred embodiment of the present invention, which, however, should not be taken to limit the invention, but are for explanation and understanding only.

In the drawings:

FIG. 1 shows an isometric view of the orthotic support device applied on a right foot, in accordance with one or more embodiments set forth herein;

FIG. 2A shows an anterior perspective view of the double strap of the orthotic support device of FIG. 1, in accordance with one or more embodiments set forth herein;

FIG. 2B shows a posterior perspective view of the double strap of the orthotic support device of FIG. 1, in accordance with one or more embodiments set forth herein;

FIG. 3 shows a posterior perspective view of the double strap of the orthotic support device of FIG. 1 connected to a compression sleeve, in accordance with one or more embodiments set forth herein;

FIG. 4 shows a posterior perspective view of the double strap of the orthotic support device of FIG. 1 with a magnified and exploded section of the compression sleeve and double strap, in accordance with one or more embodiments set forth herein;

FIG. 5 shows an anterior perspective view of the right foot with the orthotic support device of FIG. 1 with the strap system unwrapped, in accordance with one or more embodiments set forth herein;

FIG. 6 shows an anterior perspective view of the right foot with the orthotic support device with the strap system of FIG. 1 partially wrapped, in accordance with one or more embodiments set forth herein;

FIG. 7 shows a plantar view of the right foot with the orthotic support device of FIG. 1 partially wrapped, in accordance with one or more embodiments set forth herein;

FIG. 8 shows a posterior perspective view of the orthotic support device with the strap system of FIG. 1 partially wrapped, in accordance with one or more embodiments set forth herein;

FIG. 9 shows an anterior perspective view of the right foot with the orthotic support device of FIG. 1 fully wrapped, in accordance with one or more embodiments set forth herein;

FIG. 10 shows a plantar view of the right foot with the orthotic support device of FIG. 1 fully wrapped, in accordance with one or more embodiments set forth herein; and

FIG. 11 shows a posterior perspective view of the right foot with the orthotic support device of FIG. 1 fully wrapped, in accordance with one or more embodiments set forth herein.

Corresponding reference characters indicate corresponding parts throughout the several views. The exemplary embodiments set forth herein are not to be construed as limiting the scope of the invention in any manner.

### DETAILED DESCRIPTION OF THE INVENTION

The present invention will be discussed hereinafter in detail in terms of various exemplary embodiments according to the present invention with reference to the accompanying drawings. In the following detailed description, numerous specific details are set forth to provide a thorough understanding of the present invention. It will be obvious, however, to those skilled in the art that the present invention may be practiced without these specific details. In other instances, well-known structures are not shown in detail to avoid unnecessary obscuring of the present invention.

Thus, all the implementations described below are exemplary implementations provided to enable persons skilled in the art to make or use the embodiments of the disclosure and are not intended to limit the scope of the disclosure, which is defined by the claims. As used herein, the word “exemplary” or “illustrative” means “serving as an example, instance, or illustration.” Any implementation described herein as “exemplary” or “illustrative” is not necessarily to be construed as preferred or advantageous over other implementations.

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regard to the foot, the term “dorsal” refers to the top of the foot and the term “plantar” refers the bottom of the foot.

Similarly, positions or directions may be used herein with reference to anatomical structures or surfaces. For example, as the current devices, systems, and methods are described herein with reference to use with the ankle, the foot, and lower leg may be used to describe the surfaces, positions, directions or orientations of the devices, systems, instrumentation and methods. Further, the devices, systems, and methods, and the aspects, components, and features, disclosed are described with respect to one side of the body for brevity purposes. However, as the human body is relatively symmetrical or mirrored about a line of symmetry (body midline), it is hereby expressly contemplated that the devices, systems, and methods, and the aspects, components, and features, described and/or illustrated in this disclosure may be changed, varied, modified, reconfigured or otherwise altered for use or association with another side of the body for a same or similar purpose without departing from the spirit and scope of the disclosure. For example, the devices, systems, and methods, and the aspects, components, and features, described herein with respect to the right leg may be mirrored so that they likewise function with the left leg. Further, the devices, systems, and methods, and the aspects, components, and features, disclosed are described with respect to the foot, ankle, and lower leg for brevity purposes, but it should be understood that the devices, systems, and methods may be used with other limbs of the body having similar structures.

Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

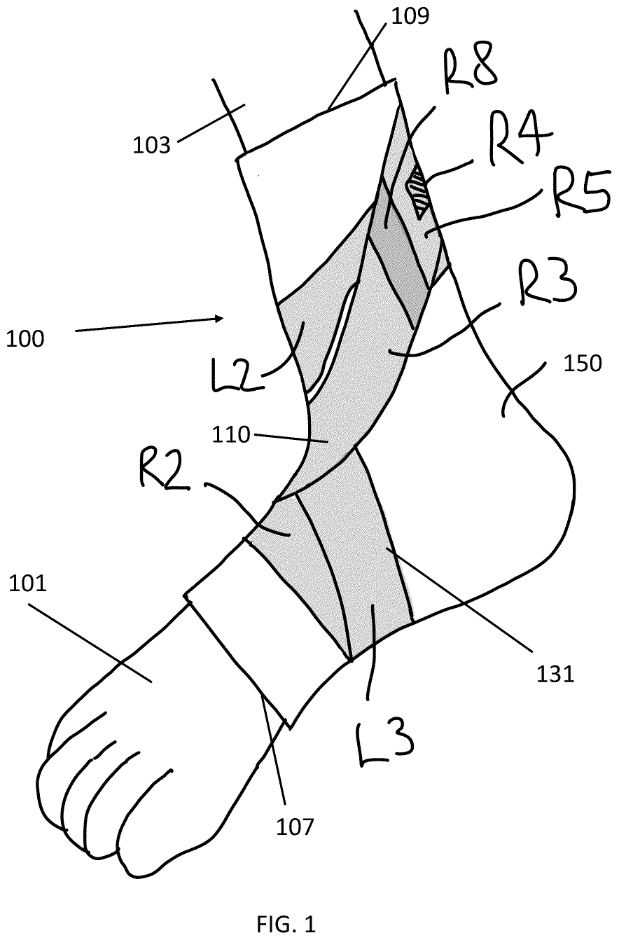

Referring to the drawings, wherein like reference numerals are used to indicate like or analogous components throughout the several views, and with particular reference to FIG. 1, an isometric view of an orthotic ankle support device 100 as applied on a right ankle of a user is shown. An ankle compression sleeve 150 is shown surrounding the ankle, extending around a foot 101 towards a first end 107 in the direction of the toes. The ankle sleeve 150 may extend around the lower leg to a second end 109. The second end 109 may extend towards a knee. The ankle sleeve 150 may have a foot aperture and a leg aperture, the two apertures configured (e.g., sized and dimensioned) to accommodate the foot, the ankle, and a portion of the lower leg, and to surround the ankle region of a person's leg. A double strap 110 is shown wrapped and secured around the sleeve 150. In certain embodiments, the ankle sleeve may have a third aperture for a heel area of a user's foot.

The ankle sleeve 150, may provide a level of compression from approximately 130 Pa to approximately 5.3 kPa. The sleeve 150 may be manufactured using any conventional technique such as, for example, circular knitting. The sleeve 150 may be made of, for example, elastane, spandex or other such high performance, lightweight, and breathable fabrics.

In an alternative embodiment of the orthotic ankle support device 100, the double strap 110 may be sewed, fused, and/or knit onto the compression ankle sleeve 150. In still another embodiment the ankle sleeve 150 may be a sock, covering the entire foot, the ankle, and/or a portion of the lower leg.

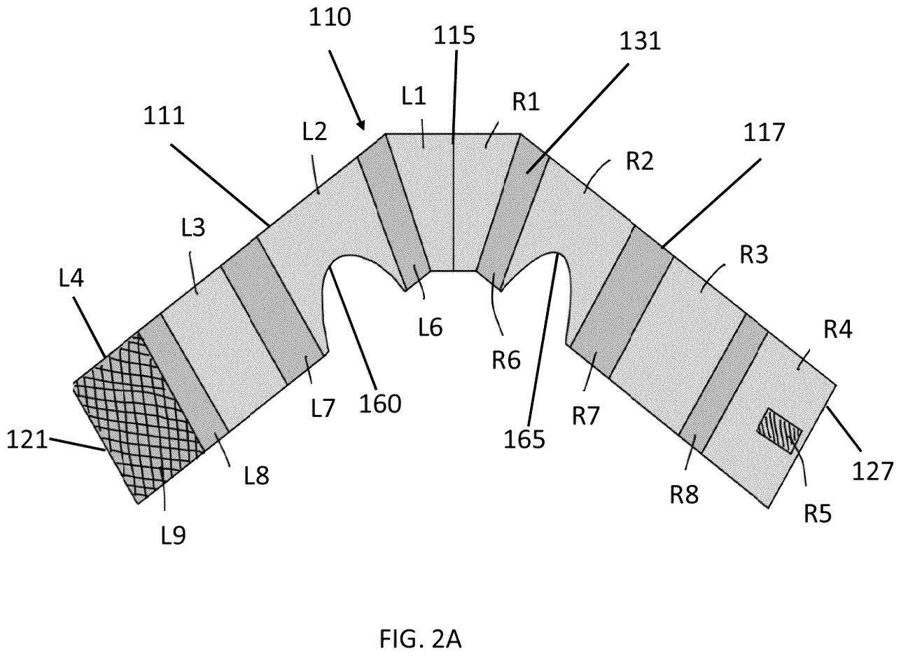

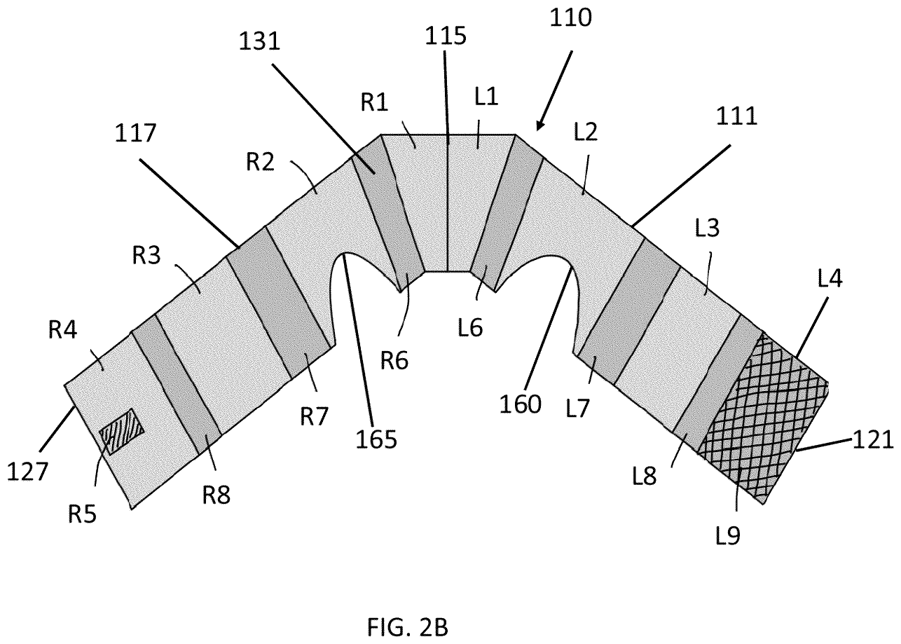

With reference to FIG. 2A, an anterior view of the double strap 110 is depicted. With reference to FIG. 2B, a posterior view of the double strap 110 is depicted. The double strap 110 may have two segmented sections, the first strap 111, or left strap, and the second strap 117, or right strap, extending from a midline 115. Thus, two straps (i.e., first strap 111 and second strap 117) are connected at midline 115, forming double strap 110. The double strap 110 may have, for example, multiple alternating segments of non-stretchable, inelastic, or rigid materials and of elastic or stretchable segments. In the first strap 111 or left strap, inelastic segments L1, L2, L3, and L4 and the stretchable segments are L6, L7, and L8 are shown in an alternating configuration, extending from the midline 115, having a free end 121 of L4. In the second strap 117 or right strap, inelastic segments R1, R2, R3, and R4 and the stretchable segments are R6, R7, and R8 are shown in an alternating configuration, extending from the midline 115 to a free end 127 of R4. R1 and L1 connect to each other at midline 115. The non-stretchable, inelastic, or rigid materials may be such that the material conforms to the user's foot, but still provides support and is not easily yielding to changes in shape and dimension.

Each of the segments (e.g., L1, L2, L3, L4, L6, L7, L8, R1, R2, R3, R4, R6, R7, and R8) have a first side facing the leg/foot and a second side facing away from the leg/foot. Segment L4 may have a first fastener component L9 on the first side. Segment L4 may have a finger loop component L5 on the second side. Segment R4 may have a second component R9 on the second side. Segment R4 may have a finger loop component R5 on the first side. First fastener component L9 may be a hook component and second fastener component R9 may be a loop component forming a hook and loop fastener when first fastener component L9 is connected with second fastener component R9. In other embodiments, first fastener component L9 may be a loop component and second fastener component R9 may be a hook component forming a hook and loop fastener when first fastener component L9 is connected with second fastener component R9.

In other embodiments, first fastener component L9 may be on the second side of segment L4. Segment L4 may have a finger loop component L5 on the first side. Second fastener component R9 may be on the first side. Segment R4 may have finger loop component R5 on the second side. First fastener component L9 may be a loop component and second fastener component R9 may be a hook component forming a hook and loop fastener when first fastener component L9 is connected with second fastener component R9.

First fastener component L9 and second fastener component R9, may be, for example, on opposite sides of double strap 110.

In embodiments where first fastener component L9 and second fastener component R9 are hook and loop fasteners, the hook component may be smaller to facilitate desired positioning connection and to help adjust strap tension.

For example, Velcro® may be used for the hook component and loop components, but other hook and loop systems may be used. First fastener component L9 and second fastener component R9 may also be opposing components of other common fasteners components to connect to each other including, for example, zippers, buttons, snaps, buckles, or other common securing mechanisms.

Finger loop component L5 and finger loop component R5 may be positioned on opposite sides of double strap 110 and may be configured (e.g., sized and dimensioned) to accommodate a finger or multiple fingers of a person applying the double strap 110, to facilitate placement and tensioning of the double strap 110. First finger loop L5 may be, for example, positioned on the side of component L4 opposite first fastener component L9. Second finger loop R5 may be, for example, positioned on the side of component R4 opposite second fastener component R9.

Referring to the first strap 111, inelastic segment L1 may extend at a first end from the midline 115 to a second end connected to a first end of stretchable segment L6. Stretchable segment L6 may be connected at a second end to a first end of inelastic segment L2. Inelastic segment L2 may be connected at a second end to a first end of stretchable segment L7. Stretchable segment L7 may be connected at a second end to a first end of inelastic segment L3. Inelastic segment L3 may be connected at a second end to a first end of stretchable segment L8. Stretchable segment L8 may be connected at a second end to a first end of segment L4. Segment L4 may extend to the free end 121 and may have first fastener component L9 on the first side (or a second side) of L4 between the first end of segment L4 and the free end 121. First finger loop L5 may be positioned on the side of L4 opposite the first fastener component L9 between the first end of segment L4 and the free end 121.

Referring to the second strap 117, inelastic material R1 may extend at a first end from the midline 115 to a second end connected to a first end of stretchable segment R6. Stretchable segment R6 may be connected at a second end to a first end of inelastic segment R2. Inelastic segment R2 may be connected at a second end to a first end of stretchable segment R7. Stretchable segment R7 may be connected at a second end to a first end of inelastic segment R3. Inelastic segment R3 may be connected at a second end to a first end of stretchable segment R8. Stretchable segment R8 may be connected at a second end to a first end of R4. Segment R4 may extends to the free end 127 and may have second fastener segment R9 on the second side (or the first side) of R4 between the first end of segment R4 and the free end 127. Second finger loop R5 may be positioned on the side of R4 opposite the second fastener component R9 between the first end of segment R4 and the free end 127.

The inelastic segments (e.g., R1, R2, R3, R4, L2, L3, and L4), provide support for the ankle, inhibiting ankle motion, particularly with foot inversion and foot eversion motion and with inversion type sprains and eversion type sprains. The elastic segments (e.g., R6, R7, R8 L6, L7, and L8), provide flexibility between the inelastic segments as a user's foot is moved and promoting for some range of motion and flexibility, but still inhibiting foot inversion and foot eversion motion by a user wearing the double strap 110 and the sleeve 150.

There may be other embodiments of the double strap 110 with a smaller or larger number of elastic and inelastic segments. The embodiment in FIGS. 2A and 2B provides a balance between ankle support and flexibility. The inelastic material may, for example, be formed of ballistic nylon, polyester, polyurethane, or other materials with similar properties. The elastic or stretchable segments may, for example, be formed of materials such as elastane, spandex, rubber, or other materials with similar properties. The inelastic and stretchable segments may, for example, be secured to each other via fusing, gluing, sewing, knitting, and/or combinations thereof.

Segment R1 and segment L1 are depicted as having a triangular or trapezoidal shape. However, in other embodiments, segments L1 and R1 may be different shapes. Segments R2, R3, R4, R6, R7, R8, L2, L3, L4, L6, L7, and L8 are depicted as being rectangular or square, but in other embodiments, they may be different shapes.

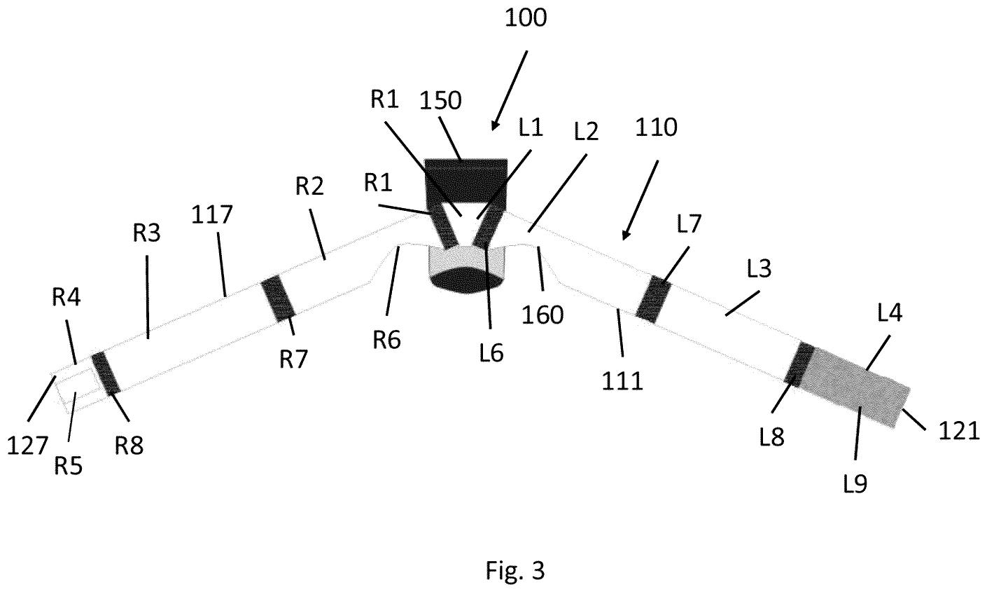

With reference to FIG. 3, a posterior view of another embodiment of the double strap 110 is depicted. The double strap 110 is shown connected to compressions sleeve 150 and the first strap 111 and the second strap 117 shows extended in the lateral and medial directions. In the first strap 111, inelastic segments L1, L2, L3, and L4 and the elastic segments are L6, L7, and L8 are shown in an alternating configuration, extending to the free end 121 of L4. In the second strap 117, inelastic segments R1, R2, R3, and R4 and the stretchable segments are R6, R7, and R8 are shown in an alternating configuration, extending to a free end 127 of R4.

Segment L4 may have a first fastener component L9 on the first side. Segment L4 may have a finger loop component L5 on the second side. Segment R4 may have a second component R9 on the second side. Segment R4 may have a finger loop component R5 on the first side. First fastener component L9 may be a hook component and second fastener component R9 may be a loop component forming a hook and loop fastener when first fastener component L9 is connected with second fastener component R9.

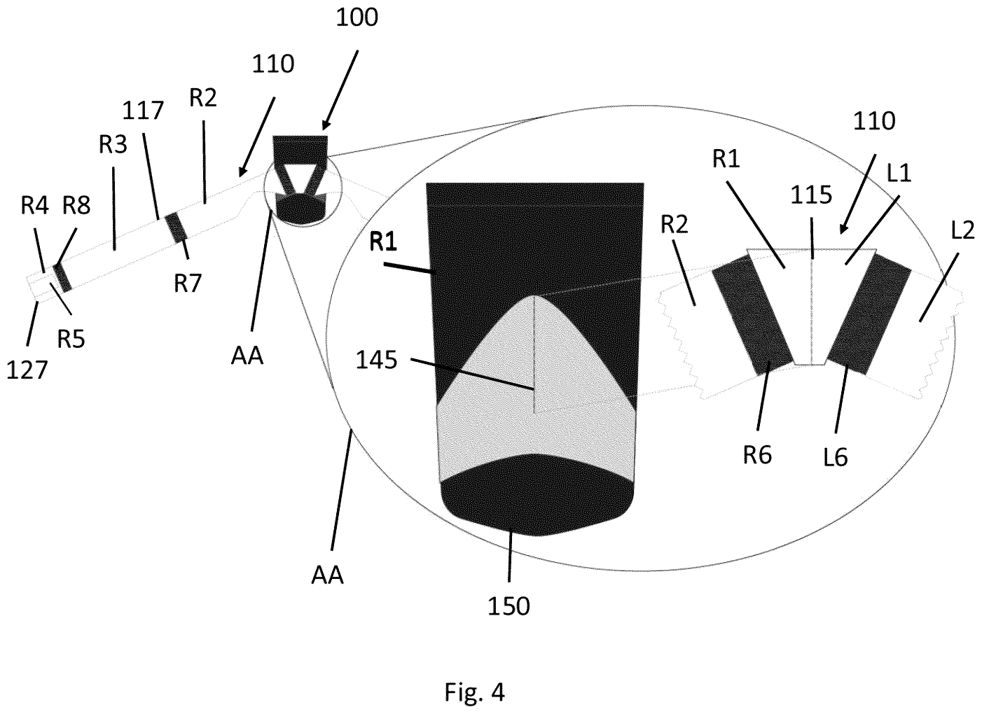

With reference to FIG. 4, a posterior view of the ankle support device 100 is depicted with section AA magnified and the magnified section AA showing the compression sleeve 150 with a connection line 145, to which midline 115 may be placed. In some embodiments, the leg facing side of midline 115 may be connected to connection line 145. Midline 115 may be connected to connection line 145 such that inelastic segment R1 and inelastic segment L1 are positioned on opposite sides of connection line, and inelastic segment R1 may be towards the medial side of sleeve 150 and L1 may be towards the lateral side of sleeve 150, or vice versa, depending on which user ankle is inserted into sleeve 150.

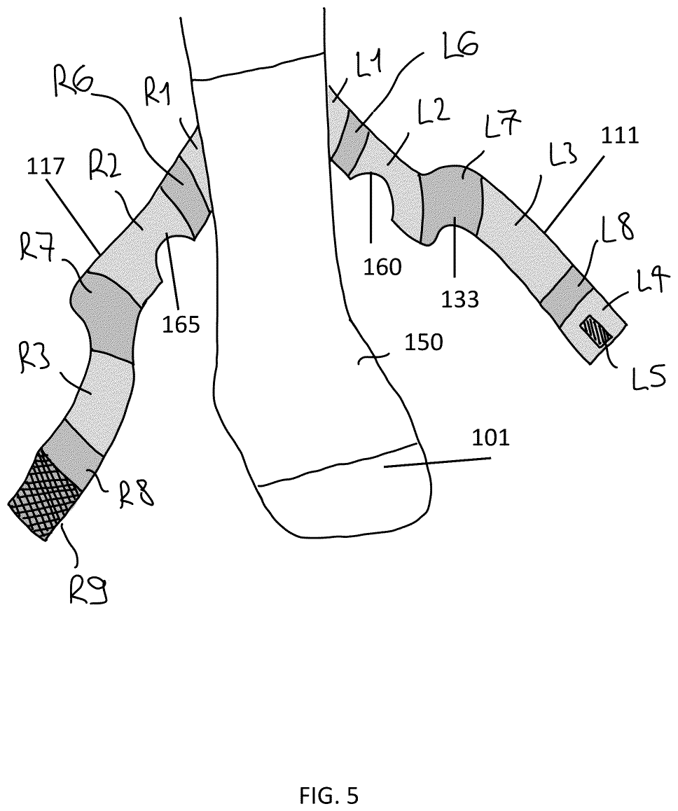

With reference to FIG. 5, the ankle support device 100 is shown with the sleeve placed around the ankle. The double strap 110 has not been wrapped around the foot yet and is depicted in a starting position for application and fastening.

With reference to FIGS. 2B and 3, double strap 110 may be connected to a posterior facing side of compression sleeve 150 such the first side of segment L1 and the first side of segment R1 may be approximately symmetrically connected at approximately the midline 115. Double strap 110 and compression sleeve 150 may be connected by fusing, gluing, sewing, knitting, and/or combinations thereof.

In certain other embodiments, double strap 110 may be detached from the compression sleeve 150 and a user may place the double strap 110 such that the midline 115 of the first side is placed against the sleeve 150, rather than connected

Double strap 110 may have indents in the inelastic segments L2 and R2 of both straps, as depicted in FIGS. 2A-3. An indent 160 may be in inelastic segment L2 and an indent 165 may be in inelastic segment R2. Indent 160 and indent 165 are depicted as being curvilinear, however other shaped indents may be used. As shown here, indents 160 and 165 are within the plantar side of inelastic segment L2 and inelastic segment R2 respectively. Indent 160 and indent 165 minimize excess material at the points of overlap between the first strap 111 and the second strap 117 of the double strap 110 during application and use. Indent 160 and indent 165 serve to inhibit bunching or piling and provides a smooth interface between the sleeve 150 and the double strap 110. Since less overlap, bunching, or piling exists once the strap is applied, bulk is minimized, and greater comfort is provided for the wearer.

In certain other embodiments (not shown), indent 160 and indent 165 may be within the dorsal side of inelastic segment L2 and inelastic segment R2 respectively. In still certain other embodiments (not shown), there may be indents on the dorsal and plantar sides of inelastic segment L2 and inelastic segment R2 respectively.

While description has been provided for the right ankle, when applied to the left ankle, the straps may be reversed (e.g., left strap 111 first followed by right strap 117).

### Method of Application

The following method of applying the orthotic ankle support device 100, the structure of which has been described with reference to FIGS. 1-3, is now made with reference to FIGS. 3-9 and is described for use on the right ankle. The steps include, placing the compression sleeve 150 around the ankle. The double strap 110 is applied by starting with the right strap 117 (when the double strap 110 is applied on the left ankle the user may start with the left strap 111).

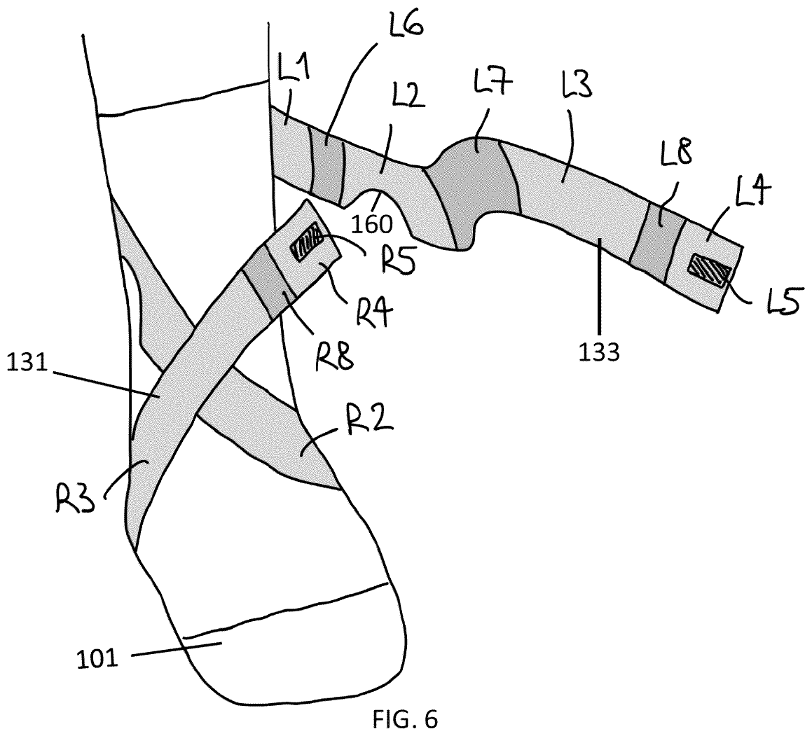

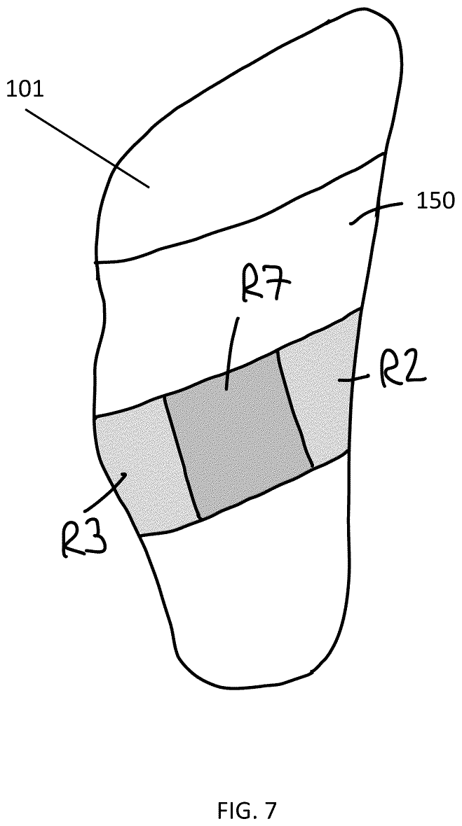

Referring to FIG. 6, the double strap 110 may be wrapped around the ankle starting from the high lateral side of the ankle and right strap 117 may be moved over the bridge of the foot 101, on the dorsal side of the foot, towards the medial side of the foot 101. With reference to FIG. 7, the right strap 117 may be pulled under the arch of the foot 101 and brought back to the lateral side of the ankle as shown in FIG. 6. The right strap 117 may be pulled up diagonally towards the medial side of the high ankle. Finger loop component R5 may assist the user in tensioning the strap by, as the user may hook a finger or fingers through R5 to adjust tension or to hold the strap in place on the posterior medial side of the ankle.

Referring to FIG. 61, segments L1 and R1 may be of triangular shape to favor the tensioning of the strap at an angle smaller than 90° with respect to the midline 115 of the double strap 110, such that the straps extend downwards or in a planter direction relative to the leg. Having segments L1 and R1 in a triangular shape positions the left strap 111 and the right strap 117 may be at angles to each other rather than being linear. Referring to FIG. 6, by having the strap at an angle, the user may fully tension the strap in the direction in which the strap is pulled as the angle may be in the direction of pull. For example, the straps may be pulled across the dorsal side of the foot towards the arch (so downwards). In an inline configuration, the straps would be tensioned around the ankle (e.g., parallel with respect to the ground) instead of being angled downward.

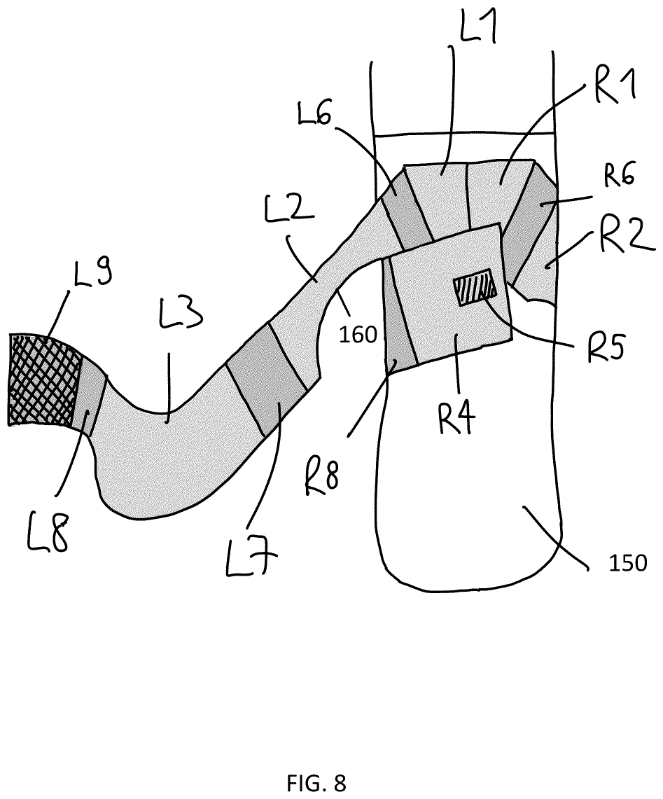

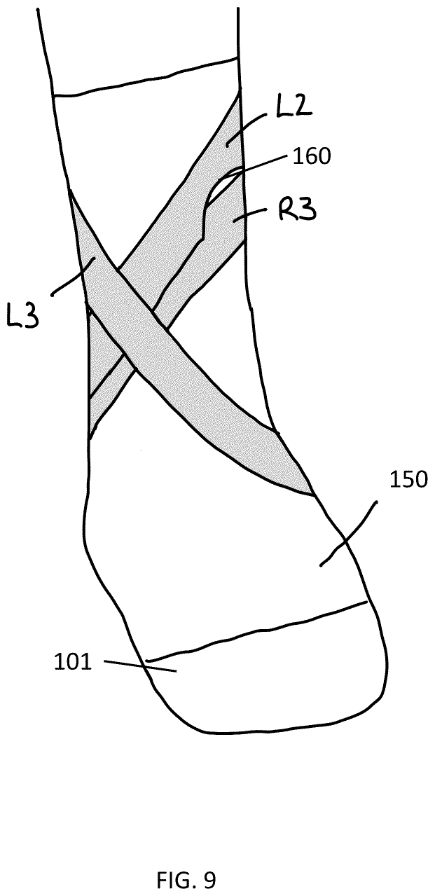

With reference to FIG. 8, after the right strap 117 is applied, the left strap 111 is wrapped around the ankle and applied over the right strap 117. While the right strap 117 is held in place on the posterior medial side of the ankle, the left strap is applied over the bridge of the foot 101, crossing over the right strap 117 and segment R3 towards the lateral side of the foot 101 as depicted in FIG. 9.

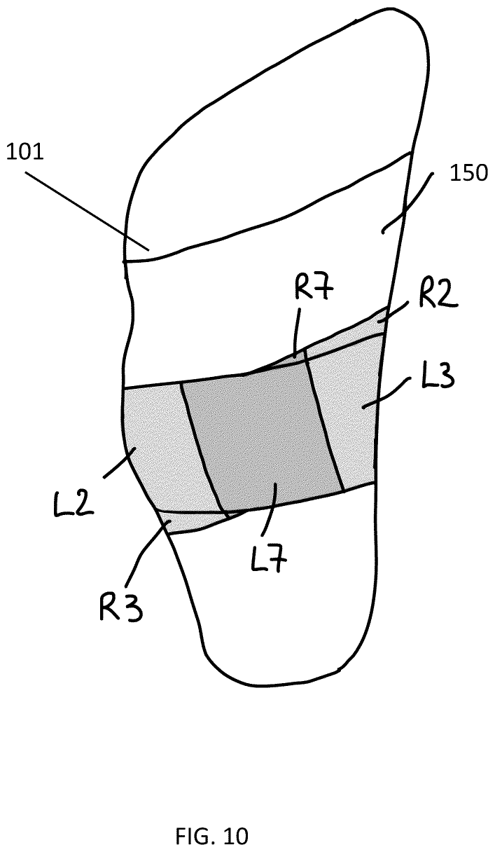

Left strap 111 and right strap 117 may have indents on L2 and R2 to promote a smooth overlap of the two sides and prevent them from bunching when strap 110 is applied (see FIGS. 4, 6, and 7). With reference to FIG. 10, the left strap 111 may be looped under the arch of the foot 101, and pulled towards the medial side of the right ankle (the loop component L5 may assist the user in the tensioning of the left strap and in holding it in place)

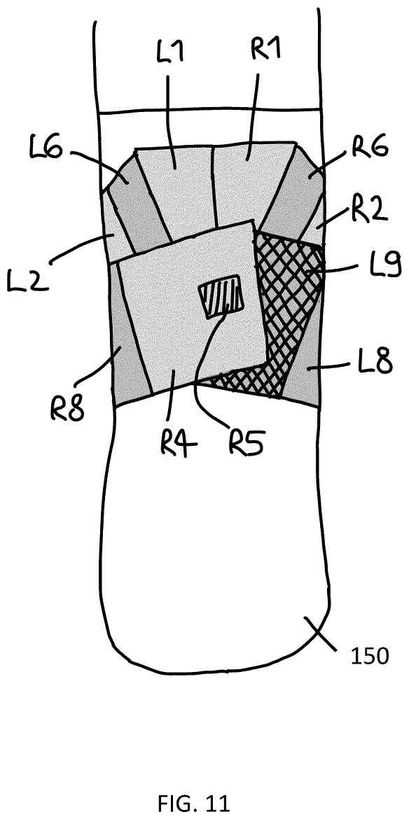

With reference to FIG. 11, the two straps (e.g., left strap 111 and right strap 117) may be secured to each other on the rear side of the ankle. Both left strap 111 and right strap 117 may have been tensioned by the user and their ends (e.g., free end 121 and free end 127) may be located towards the posterior of the ankle. The user may then pull left strap 111 and right strap 117 together to apply increased tension and lock them to each other via the hook and loop fastener (e.g., first fastener component L9 and second fastener component R9). The first fastener component L9 and the second fastener component R9 may be present on opposite sides R4 and L4 respectively such that the hook and loop components are facing and fastenable.

As depicted in FIG. 11, second fastener component R9 may be placed over first fastener component L9, such that the left strap 111 may be directly positioned against the calf portion of the user's leg and the right strap 117 may be connected to the left strap. Second fastener component R9 is shown with the hook portion and first fastener component L9 is shown with the loop portion, such that placing the two together forms a fastened connection.

This hook and loop fastening system is arranged to be oriented approximately perpendicularly to the calf direction (or ground if the user is standing) as depicted in FIG. 11. Thus, additional materials are not required to hold the double strap system in place.

The advantage is that this orthotic ankle support device 100 provides the support that is characteristic of bulkier and more rigid ankle braces, while being thinner and lighter.

While description has been provided for the right ankle, when applied to the left ankle, the user may apply the straps in a reverse order (e.g., left strap 111 first followed by right strap 117).

A method has been described where the right strap is applied first to the right foot, applying the left strap first nonetheless provides ankle support. However, wrapping the right strap 117 first provides direct pressure on the foot and ankle, supporting the outer part of the foot. By wrapping the right strap 117 first there may be greater inhibition towards foot inversion and inversion type sprains. Inversion sprains are the most common for athletes. Even if the left strap is first applied, the foot and ankle are supported, and inversion sprains may still be inhibited.

For users more concerned with eversion types of sprains, applying the left strap first provides direct pressure on the foot and ankle, supporting the inner part of the foot, thus inhibiting foot eversion and eversion type sprains.

The method may be applied to the left foot, with the left strap 111 being applied first and the right strap 117 being applied second, for users concerned with inversion type sprains. The right strap 117 may be applied first for users concerned with eversion type sprains.

The methods of use described are preferred embodiments for the order and positioning of the straps. However, the order and positioning may be adjusted to conform to the user's foot, leg, and ankle. There are still orders and positioning which may provide support or minimize sprains.

The advantages of the orthotic ankle support device 100 over other ankle support products include: a double strap 110 having embedded stretchable segments between inelastic segments providing ankle support in conjunction with a thin compression sleeve 150. The combination of both the right strap 117 and the left strap 111 provides protection for the ankle and inhibits inversion and plantar flexion sprains common in competitive sport settings. Combining the double strap 110 alternating inelastic and stretchable segments with the compression sleeve 150 provides a snug support, while reducing bulk and weight of the brace. Indents (e.g., indent 160 of segment L2 and indent 165 of segment R2) inhibit bunching and wrinkling of the double strap 110 upon applied.

For application on a left foot, the double strap 110 may be symmetric, with the inelastic and stretchable segments arranged in a symmetric manner with respect to the midline 115 of the straps secured to the ankle sleeve on the rear part of the foot.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An orthotic ankle support system comprising:
a compression sleeve having an interior space to receive and conform to a foot, an ankle, and a portion of a lower leg of a person;
a double strap comprising:
a midline;
a first strap comprising a first inelastic segment extending from the midline towards a last inelastic segment at a free end, and having a plurality of alternated elastic segments and inelastic segments, therebetween;
a second strap comprising, a first inelastic segment extending from the midline towards a last inelastic segment at a free end, and having a plurality of alternated elastic segments and inelastic segments therebetween;
the first strap and the second strap extending from the midline in opposing directions;
the midline positioned on a posterior side of the compression sleeve having the first strap and the second strap wrapped in opposite directions around the compression sleeve and fastened to each other to hold and grip the foot and ankle, when inserted into the sleeve.

2. The orthotic ankle support system of claim 1, wherein the midline is connected to the posterior side of the compression sleeve and on the posterior of a user's foot.

3. The orthotic ankle support system of claim 1, wherein the first strap and the second strap are symmetrically angled, about the midline in a plantar direction.

4. The orthotic ankle support system of claim 3, wherein the first inelastic segment of the first strap is selected from a triangular or trapezoidal shape and the first inelastic segment of the second strap is selected from a triangular or trapezoidal shape.

5. The orthotic ankle support system of claim 1, wherein the first strap further comprises:
a first elastic segment connected to and between the first inelastic segment and a second inelastic segment;
a second elastic segment connected to and between the second inelastic segment and a third inelastic segment; and
a third elastic segment connected to and between the third inelastic segment and the last inelastic segment; and wherein
the second strap further comprises:
a first elastic segment connected to and between the first inelastic segment and a second inelastic segment;
a second elastic segment connected to and between the second inelastic segment and a third inelastic segment; and
a third elastic segment connected to and between the third inelastic segment and the last inelastic segment.

6. The orthotic ankle support system of claim 1, wherein one of the plurality of inelastic segments of the first strap has an indent on the plantar side of the one inelastic segment; and one of the plurality of inelastic segments of the second strap has an indent on the plantar side of the one inelastic segment.

7. The orthotic ankle support system of claim 5, wherein the second inelastic segment of the first strap and a concave indent on the plantar side and the second inelastic segment of the second strap and a concave indent on the plantar side.

8. The orthotic ankle support system of claim 1, wherein the compression sleeve provides from 130 Pa to 5.3 kPa of compressive force.

9. The orthotic ankle support system of claim 1, wherein motion of the ankle is inhibited by the first inelastic segment, the last inelastic segment and the plurality of inelastic segments therebetween, of the first strap; and the first inelastic segment, the last inelastic segment and the plurality of inelastic segments therebetween, of the second strap inhibit ankle motion.

10. The orthotic ankle support system of claim 9, wherein a range of motion of the ankle is promoted but foot inversion and foot eversion are inhibited.

11. The orthotic ankle support system of claim 1, wherein the first strap has a first fastener component on the last segment and the second strap has a second fastener component on the last segment, the first fastener component and the second fastener component fastened to each other.

12. The orthotic ankle support system of claim 11, wherein the first fastener component and the second fastener component for a hook and loop fastener.

13. The orthotic ankle support system of claim 1, wherein the last segment of the first strap has a first finger loop configured for inserting a finger, and the last segment of the second strap has a second finger loop configured for inserting a second finger, the first loop and the second loop configured to promote first strap and second strap adjustment.

14. The orthotic ankle support system of claim 5, wherein
the first strap is positioned about the foot and ankle such that;
the first strap extends along a high lateral section of the ankle;
the first strap extends over a bridge of the foot towards a medial side of the foot;
the first strap extends under an arch of the foot towards a lateral side of the foot;
the first strap extends a second time over the bridge of the foot towards a high medial section of the ankle; and
the second strap is positioned about the foot and ankle such that;
the second strap extends along the high medial section of the ankle;
the second strap extends over the bridge of the foot towards the lateral side of the foot;
the second strap extends under the arch of the foot towards the medial side of the foot;
the second strap extends over the bridge of the foot towards the high lateral section of the ankle; and
wherein the first strap and the second strap are affixed to each other adjacent to the midline.

15. A method for applying an orthotic ankle support device comprising:
applying a compression sleeve to a foot, an ankle, and a lower leg, the compression sleeve having a double strap connected at a midline to a posterior facing section of the compression sleeve;
moving a first strap about a high lateral section of the ankle;
moving the first strap over a bridge of the foot towards a medial side of the foot;
moving the first strap under an arch of the foot towards a lateral side;
moving the first strap over the bridge of the foot towards a high medial section of the ankle;
holding the first strap to the midline of the double strap;
moving a second strap section about the high medial section of the ankle;
moving the second strap over the bridge of the foot towards the lateral side of the foot;
moving the second strap under the arch of the foot towards the medial side;
moving the second strap over the bridge of the foot towards a high lateral section of the ankle;
adjusting the strap tension; and
affixing the second strap to the first strap.

16. An orthotic ankle support device comprising:
a double strap comprising:
a midline;
a first strap having alternated inelastic segments and elastic segments, a first inelastic segment extending from the midline towards a last inelastic segment at a free end;
a second strap extending from the midline opposite the first strap, the second strap having alternated inelastic segments and elastic segments, a first inelastic segment extending from the midline towards a last inelastic segment at a free end;
the double strap positioned around an ankle having the first strap and the second strap wrapped in opposite directions and adjusted to vary tension to hold and grip the ankle.

17. The orthotic ankle support system of claim 16, further comprises a pressure sleeve surrounding the ankle.

18. The orthotic ankle support system of claim 16, wherein the first strap and the second strap are symmetrically angled, about the midline in a plantar direction.

19. The orthotic ankle support system of claim 18, wherein one of the plurality of inelastic segments of the first strap has an indent on the plantar side of the one inelastic segment; and one of the plurality of inelastic segments of the second strap has an indent on the plantar side of the one inelastic segment.

20. The orthotic ankle support system of claim 16, wherein motion of the ankle is inhibited by the first inelastic segment, the last inelastic segment and the plurality of inelastic segments therebetween, of the first strap; and the first inelastic segment, the last inelastic segment and the plurality of inelastic segments therebetween, of the second strap inhibit ankle motion.

21. The orthotic ankle support system of claim 16, wherein the first strap has a first fastener component on the last segment and the second strap has a second fastener component on the last segment, the first fastener component and the second fastener component fastened to each other.

22. The orthotic ankle support system of claim 16, wherein the last segment of the first strap has a first finger loop configured for inserting a finger, and the last segment of the second strap has a second finger loop configured for inserting a second finger, the first loop and the second loop configured to promote first strap and second strap adjustment.

23. The orthotic ankle support system of claim 20, wherein
the first strap is positioned about the foot and ankle such that;
the first strap extends along a high lateral section of the ankle;
the first strap extends over a bridge of the foot towards a medial side of the foot;
the first strap extends under an arch of the foot towards a lateral side of the foot;
the first strap extends a second time over the bridge of the foot towards a high medial section of the ankle; and
the second strap is positioned about the foot and ankle such that;
the second strap extends along the high medial section of the ankle;
the second strap extends over the bridge of the foot towards the lateral side of the foot;
the second strap extends under the arch of the foot towards the medial side of the foot;
the second strap extends over the bridge of the foot towards the high lateral section of the ankle; and
wherein the first strap and the second strap are affixed to each other adjacent to the midline.
